(19) United States
(12) Patent Application Publication
(10) Pub. No.: US 2022/0266017 A1
(43) Pub. Date: Aug. 25, 2022

(54) Systems and Methods For Movement Modulation of a Body Part of a Subject

(71) Applicant: UNIVERSITÉ DE MONTRÉAL, Montrèal (CA)

(72) Inventors: Marco Bonizzato, Quèbec (CA); Marina Martinez, Quèbec (CA)

(73) Assignee: UNIVERSITÉ DE MONTRÉAL, Montrèal, QC (CA)

(21) Appl. No.: 17/631,210

(22) PCT Filed: Jul. 30, 2020
(86) PCT No.: PCT/CA2020/051047

§ 371 Date: Jan. 28, 2022

Related U.S. Application Data
(60) Provisional application No. 62/880,364, filed on Jul. 30, 2019.

(51) Int. Cl. A61N 1/36

(52) U.S. Cl. CPC A61N 1/36067; A61N 1/36139

(57) ABSTRACT

A method for modulating a movement of a body part comprising: acquiring movement event data relating to the body part during a movement event, the movement event data including: a movement event parameter requiring modulation, a trigger value representative of a first time point in the movement event, and a target value representative of desired movement at a second, later, time point in the movement event; acquiring in real-time current movement data associated with a current movement event of the body part, the current movement data including current values of the movement event parameter; analyzing the current movement data to determine presence of the trigger value in the current movement event; and in response to identification of the trigger value in the current movement data, causing a brain stimulation assembly to apply a predetermined modulation signal to a brain motor region of the subject to cause the modulation of the body part towards the target value at the second time point in the current movement event.

### FIELD

The present technology relates to systems and methods for movement modulation of a body part of a subject, and more specifically to systems and methods of movement modulation of a body part of a subject by neurostimulation.

### BACKGROUND

Movement disorders range from paralysis to reduced motor control in a body part of a subject. Examples of movement disorders include those affecting the limbs of a subject, such as the legs, arms, hands and feet.

For movement disorders such as incomplete spinal cord injuries, a partial recovery of normal voluntary movement in the affected body part may be possible through voluntary movement efforts by the subject. For example, a partial recovery of voluntary movement of a subject's legs affected by an incomplete spinal injury may be possible through assisted training on a treadmill and over-ground. As an another example, a partial degree of control of the subject's hand to grasp and handle daily-living objects may be possible through assisted hand and arm exercises.

However, in most cases, this is a very slow process, requiring months if not years of physical training, and recovery of voluntary movement of the affected body part is often unsatisfactory.

Spinal stimulation has been proposed for treating some movement disorders, particularly those of the legs. Spinal stimulation can induce an uncomfortable sensory experience to the user, including paresthesia, and does not guarantee long term effects of the treatment.

Brain neurostimulation has been proposed for treating some movement disorders. Current neurostimulation methods involve deep brain stimulation using electrical signals from an implantable device. These methods are invasive and often have side effects such as pain and unexpected impact on other movements. Deep brain stimulation methods also tend to be non-targeted which has its associated drawbacks.

It is an object of the present technology to ameliorate at least some of the inconveniences present in the prior art.

### SUMMARY

Embodiments of the present technology have been developed based on developers' appreciation of certain shortcomings associated with the existing systems and methods for treating movement disorders.

For example, in U.S. Pat. No. 6,484,059, a deep brain lead is used to stimulate the GPi or other deep brain target to treat neurological disorders such as Parkinson's Disease. The motor cortex can be used as a feedback target and both the motor cortex and the deep brain target can be stimulated concurrently. The position of the deep brain target lead is adjusted during the surgical procedure until the optimum position is found which is when the affected body portion is made to move.

In U.S. Pat. No. 5,683,422, techniques for stimulating the brain to reduce the effects of neurodegenerative disorders by means of an implantable signal generator and electrode. A sensor is used to detect the symptoms resulting from the disorder. A microprocessor algorithm analyzes the output from the sensor in order to regulate the stimulation delivered to the brain.

In U.S. Pat. No. 6,356,784 an implantable signal generator and electrode and/or an implantable pump and catheter is disclosed for providing high electrical stimulation pulses and/or drug therapy to the Pedunculopontine Nucleus (PPN). A sensor may be used to detect various symptoms of the movement disorders and the stimulation and/or drug therapy adjusted accordingly.

In US 2018/0280700, a neuromodulation system comprising a sensing unit, a stimulation unit, a central nervous system stimulation module for providing CNS stimulation, and a peripheral nervous system stimulation module for providing PNS stimulation is described.

In US 2018/0093093, a closed loop system is disclosed for real-time control of epidural and subdural electrical stimulation of the spinal cord. Sensors continuously acquire feedback signals of motion from the subject, and a signal processing device operates real-time automatic control algorithms to process the feedback signals and provide new stimulation parameters.

Broadly, developers have determined a method and a system of movement modulation of a body part of a subject, for treatment of a movement disorder or otherwise, that in certain embodiments, compared to existing neuromodulation and other movement disorder treatment approaches, has one or more of the following advantages: (i) less invasive, (ii) less complex, (iii) can be initiated at an early time meaning that a faster treatment outcome may be obtained, (iv) is not painful, (v) does not have side effects on motion of other body parts, and (vi) can lead to permanent improvement of voluntary control of the body part through brain re-learning.

From one aspect, there is provided a method for modulating a movement of a body part of a subject, the method executable by a processor of a computer system, the method comprising: acquiring, by the processor, movement event data relating to movement of the body part of the subject during a movement event, the movement event data including: at least one movement event parameter requiring modulation, a trigger value representative of a first time point in the movement event, and a target value representative of desired movement at a second time point in the movement event parameter, the second time point occurring after the first time point; acquiring in real-time, by the processor, current movement data associated with a current movement event of the muscle of the body part of the subject, the current movement data including current values of the movement event parameter; analyzing, by the processor, the current movement data to determine presence of the trigger value in the current movement event; and in response to identification of the trigger value in the current movement data, causing a brain stimulation assembly, operatively connected to the processor, to apply a predetermined modulation signal to tissue of a brain motor region of the subject to cause the modulation of the movement of the body part towards the target value at the second time point in the current movement event. The modulation of the movement of the body part occurs in real-time.

In certain embodiments, the target value comprises an up-regulation of a current motor output of the muscle of the body part of the subject.

In certain embodiments, the motor output comprises a contraction of the muscle of the body part, or a series of contractions of the muscle of the body part.

In certain embodiments, the body part is a leg, the current movement event is a phase of a gait, and the motor output is a distance of lift of a leg of the subject during the phase of the gait.

In certain embodiments, the trigger value and/or the first time point is associated with one or more of: an onset of a leg lift or a contact phase of the gait, a swing phase of the leg; and a time point between a contact phase of a foot of the leg and a contact phase of a contralateral foot of a contralateral leg.

In certain embodiments, the body part is one or more of: an arm, a leg, a neck, a trunk, a hand, a foot, a finger, and a toe.

In certain embodiments, the movement event is one or more of: walking, running, gripping, standing, and swallowing. In certain embodiments, the body part is a hand, the current movement event is a hand grip, and the motor output is a closing of fingers of the hand.

In certain embodiments, the subject has an incomplete spinal cord injury.

In certain embodiments, the movement to be modulated comprises a foot dragging or a reduced foot lift.

In certain embodiments, the processor is arranged to cause the brain stimulation assembly to apply the predetermined modulation signal at a predetermined time from identifying the trigger value in the current movement event, or the processor is arranged to cause the brain stimulation assembly to apply the predetermined modulation signal until the target value of the movement is detected.

In certain embodiments, the predetermined time is between about 0 seconds and 1 second from identifying the trigger value.

In certain embodiments, the brain motor region of the subject comprises one or more of: a cortical motor region of the brain; a deep brain motor region; and the brainstem reticular formation.

In certain embodiments, the predetermined modulation signal comprises an electrical signal delivered through an intracortical, deep brain, epidural or transcranial electrical interface, or a magnetic signal.

In certain embodiments, the predetermined modulation signal comprises a pulsed electrical signal. The pulses may be one or more of: a cathodic pulse, an anodic pulse, and a biphasic pulse. One or more pulses may be about 50 to about 400 μs/phase.

In certain embodiments, a frequency of the pulse of the predetermined modulation signal is about 100 Hz to about 400 Hz.

In certain embodiments, a length of the predetermined modulation signal is about 40 to about 500 ms.

In certain embodiments, an amplitude of the pulse is about 5 μA to about 2 mA.

In certain embodiments, the body part is a lower limb, and the predetermined modulation signal is a biphasic pulsed signal comprising 200 μs/phase, at an emission frequency of 300 Hz, having a length of about 100 ms long, and having an amplitude of about 40 μA.

In certain embodiments, the body part is an upper limb, and the predetermined modulation signal is a biphasic pulsed signal comprising 200 μs/phase, at an emission frequency of 300 Hz, having a length of about 100 ms long, and having an amplitude of about 20 μA.

In certain embodiments, the predetermined modulation signal comprises a chain of 14 pulses in 40 milliseconds.

In certain embodiments, the current movement data is acquired from an electromyographic signal associated with the muscle of the body part of the subject, the method further comprising acquiring the electromyographic signal.

In certain embodiments, the current movement data comprises image data associated with the current movement, the method further comprising obtaining the image data.

In certain embodiments, the current movement data comprises brain signal data associated with the subject, the method further comprising obtaining the brain signal data.

In certain embodiments, the current movement data comprises acceleration data associated with the current movement, the method further comprising obtaining the acceleration data.

In certain embodiments, the method further comprises determining, for the given subject, one or more of: the movement event, the trigger value, and the target value.

In certain embodiments, the method further comprises determining, for the given subject, the predetermined modulation signal, the determining the predetermined modulation signal comprising applying a preliminary modulation signal to the tissue of the brain motor region, and adjusting one or more of: an amplitude, a frequency, a duration, and a pulse duration of the preliminary modulation signal until the target value in the movement event is obtained.

In certain embodiments, the method further comprises storing in a non-transitory computer readable medium, one or more of the movement event, the trigger value, the target value, and the predetermined modulation signal.

In certain embodiments, the method further comprises acquiring, from a non-transitory computer readable medium, one or more of: the movement event, the trigger value, the target value, and the predetermined modulation signal.

In certain embodiments, the method further comprises applying, to the subject, a stimulation signal to a spinal cord of the subject.

In certain embodiments, the method further comprises executing daily the steps of acquiring in real-time, by the processor, the current movement data associated with the current movement event of the muscle of the body part of the subject; the analyzing, by the processor, the current movement data to determine presence of the trigger value in the current movement event; and in response to identification of the trigger value in the current movement data, causing the brain stimulation assembly to apply the predetermined modulation signal to tissue of a brain motor region of the subject to cause the modulation of the muscle of the subject movement towards the target value from the trigger value in the current movement event.

In certain embodiments, the method further comprises tuning the predetermined modulation signal for the subject, the tuning the predetermined modulation signal comprising: adjusting one or more of: an amplitude, a frequency, a duration, a pulse duration, and an interval time of the predetermined modulation signal responsive to a measured value of the movement event parameter at the second time point.

From another aspect, there is provided a system for modulating a movement of a body part of a subject, the system comprising: a sensor assembly, operably communicable with a processor of a computer system, arranged to sense in real-time, current movement data associated with a current movement event of a muscle of the body part of the subject; a brain stimulation assembly, operably communicable with the processor of the computer system, for applying a predetermined modulation signal to tissue of a brain motor region of the subject; the computer system having the processor arranged to execute a method comprising: acquiring an indication of a movement event of the muscle of the body part of the subject, the movement event defined by at least one movement event parameter, the at least one movement event parameter including: a trigger value representative of a first time point in the movement event, and a target value representative of a second time point in the movement event, the second time point occurring after the first time point; acquiring the current movement data from the sensor assembly and analyzing the current movement data to determine presence of the trigger value in the current movement event; and in response to identification of the trigger value in the current movement data, causing the brain stimulation assembly to apply the predetermined modulation signal to tissue of a brain motor region of the subject to cause the modulation of the muscle of the subject movement towards the target value from the trigger value in the current movement event.

In certain embodiments, the sensor assembly comprises one or more of: an electromyograph, a camera, an infrared sensor, a brain signal detector, and an accelerometer.

In certain embodiments, the brain stimulation assembly is an implantable device.

In certain embodiments, the implantable device comprises a body portion, and electrodes extending from the body portion and which are contactable with the brain tissue for applying the predetermined modulation signal to the brain tissue.

In certain embodiments, the implantable device is sized and shaped to be positioned subcutaneously in the subject.

In certain embodiments, the implantable device includes one or more of: a power supply, a transmitter, a receiver, and a processor.

From another aspect, there is provided a method of treatment of a movement disorder in a mammal with an incomplete spinal injury, the method according to any of the steps described above or any combination of these steps.

From a yet further aspect, there is provided a method for modulation of a movement of a body part of a subject during a movement event by applying modulation signals to a motor control portion of the subject's cortex at an onset of the movement event, or at a time before the onset of the movement event.

In certain embodiments, the left hand side of the brain is stimulated to control the right hand side of the body, and vice versa. Stimulation of each side of the brain can be used to control both sides of the body.

Unlike prior art systems, embodiments of the present technology movement induce the movement modulation of the body part as a direct and immediate consequence of neuromodulation. Movement modulation is induced in real-time relative to the neuromodulation. By immediate consequence is meant a measurable effect happening in a timeframe of less than a second from stimulation. The modulation of movement is based on existing neurological pathways, although long term consequences may involve creating new pathways.

Furthermore, unlike prior art systems, embodiments of the present technology comprise inducing or guiding a motor function.

Furthermore, embodiments of the present technology can achieve body part movement modulation through brain neurostimulation only. Stimulation of other body parts, such as the spinal cord, peripheral nerves or muscles, is not required.

In certain embodiments of the present technology, analysis of the current movement data is minimal. Only the trigger value is determined, in certain embodiments, without further analysis required.

In the context of the present specification, unless expressly provided otherwise, a computer system may refer, but is not limited to, an “electronic device”, an “operation system”, a “system”, a “computer-based system”, a “controller unit”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a schematic illustration of a movement of a foot during gait, to which certain embodiments of the methods and systems of the present technology can be applied.

FIG. 2 is a system for modulating a movement of a body part, the system including a computer system, a sensor assembly and a brain stimulation assembly, according to certain embodiments of the present technology.

FIG. 3 is a computing environment of the computer system of FIG. 2, according to certain embodiments of the present technology.

FIG. 4 is a schematic illustration of the sensor assembly of FIG. 2, according to certain embodiments of the present technology.

FIG. 5A is a top plan view of a subject's heading and showing an illustration of the brain stimulation assembly of FIG. 2 implanted subcutaneously in the subject's head, according to certain embodiments of the present technology.

FIG. 5B is a schematic illustration of the brain stimulation assembly of FIG. 2, according to certain embodiments of the present technology.

FIG. 6A is a schematic illustration of the system of FIG. 2 embodied as an implantable brain stimulation assembly, a sensor assembly and an implantable processor device, according to certain embodiments of the present technology.

FIG. 6B is a schematic illustration of the system of FIG. 2 embodied as an implantable brain stimulation assembly, a sensor assembly and an implantable processor device, according to certain embodiments of the present technology.

FIG. 7 is a diagram of a method for modulating a movement of the body of the subject, according to certain embodiments of the present technology.

FIGS. 8A-B illustrate modulation of a movement of a leg of a rat with incomplete spinal cord injury, according to certain embodiments of the present technology (Example 1).

FIGS. 9A-D illustrate intracortical stimulation delivery in intact rats showing phase-dependent effects of stimulation and effect of intracortical modulation amplitude, according to certain embodiments of the present technology (Example 6).

FIGS. 10A-E illustrate intracortical stimulation delivery in rats with incomplete spinal cord injury, according to certain embodiments of the present technology in which right cortex stimulation modulates left leg flexion via residual descending projections (Example 7).

FIGS. 11A-F illustrate modulating movement of a leg of a rat with incomplete spinal cord injury (Example 8), according to certain embodiments of the present technology.

FIGS. 12A-B illustrates chronic recovery of leg motor after spinal cord injury in the rat, according to certain embodiments of the present technology (Example 9).

FIGS. 13A-B illustrate leg flexion modulation during bipedal locomotion in intact rats and rats with spinal cord injury and FIG. 13C illustrates improvement of functional walking on a ladder in rats with spinal cord injury (Example 10), according to certain embodiments of the present technology.

FIGS. 14A-B illustrate step height during the swing phase of (A) intact rats, and (B) rats with a movement disorder with and without modulation, according to embodiments of the present technology (Example 11).

FIG. 15 shows ipsilateral modulation of locomotion in rats (Example 12), according to embodiments of the present technology.

FIGS. 16A-B illustrate bilateral modulation of locomotion in rats with a spinal cord injury (Example 13), according to embodiments of the present technology.

FIGS. 17A-B illustrate bilateral modulation of locomotion in cats with a spinal cord injury (Example 14), according to embodiments of the present technology.

It should be noted that, unless otherwise explicitly specified herein, the drawings are not to scale.

### DETAILED DESCRIPTION

Certain aspects and embodiments of the present technology are directed to methods and systems for modulating a movement of a body part of a subject. More specifically, in certain embodiments, the modulation is tailored to a given movement event of the subject.

Movement events may comprise any voluntary or involuntary movement of the body part for a given function, such as but not limited to: movement in one or more legs of the subject for gait; clenching of fingers of the subject for gripping; moving fingers of the subject for controlling a mouse/joystick/buttons; thrusting of the leg of the subject during kicking. The body part can be any part of the body of the subject, such as one or more of: a leg, a foot, an arm, a hand, a neck, a head, a face, a mouth, fingers, toes, a chest, and the like.

Uses of embodiments of the present technology to modulate body part movements include lessening a severity of a movement disorder, or treating a movement disorder of the subject. Movement disorders may be caused by trauma, such as incomplete spinal injuries (anatomically or clinically incomplete), by diseases affecting motor control such as Parkinson's, or by any other cause.

Non-limiting examples of voluntary motor control affectation through movement disorders comprise: limping during gait, foot dragging during gait, reduced foot lift during gait, incomplete hand clenching during gripping, shallow breathing, limited mobility of the neck when head turning, inability to grip the hand, etc.

More generally, certain embodiments of the present technology are well suited to movement disorders in which the disorder is a reduced motor output during the movement event. In certain embodiments, the present technology can be used to augment the motor output of the subject, towards a target value, during the movement event.

A movement event may be defined by one or more parameters representative of the movement of the body part. The movement event parameters may define the movement in terms of a phase/time point of the movement in the movement event, a position of the body part, a positional change of the body part, a distance of the body part from a reference point, an acceleration or deceleration of the body part, a muscle action associated with the movement.

Movement event parameters associated with the position of the body part and the positional change of the body part include, but are not limited to, coordinates and vectors. Movement event parameters associated with muscle action associated with the movement include, include but are not limited to, electrical signals from the muscles associated with the movement, or electrical signals associated with the brain of the user and relating to the movement. The electrical signals may be those as measured using EMG (electromyography).

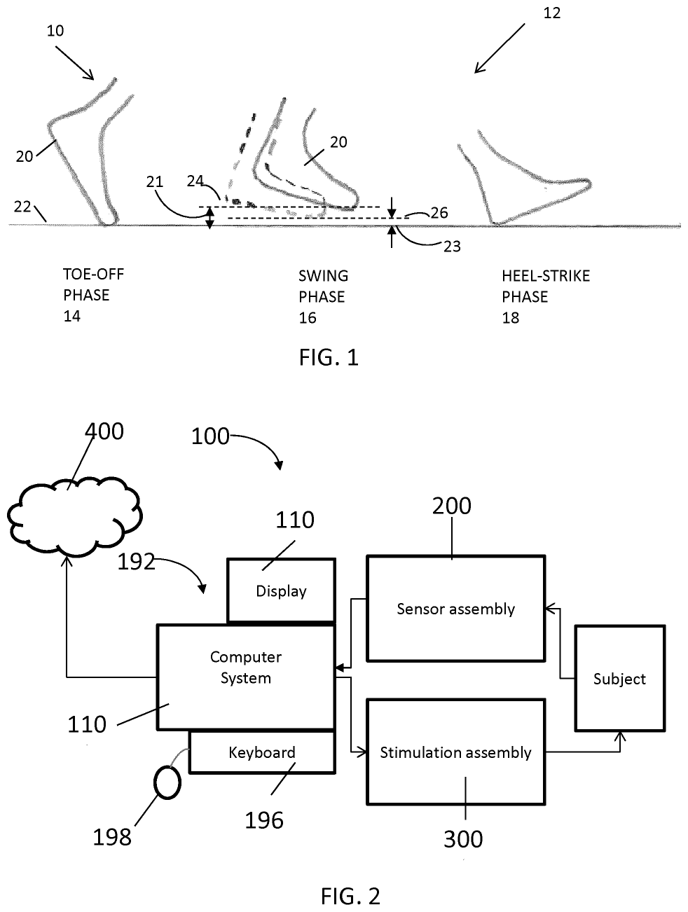

Referring to FIG. 1 and for ease of explanation, embodiments of the methods and systems of the present technology will be described below in relation to modulating a movement of a leg 10 of a subject during a movement event 12 which is walking (“gait”). The movement event 12 comprises a toe-off phase 14, a swing phase 16 and a heel strike phase 18. The movement event parameter requiring modulation comprises a distance 21 of a foot 20 of the subject from a floor 22 during the swing phase 16 (also referred to as “foot lift”). The movement disorder of the subject can be said to be a foot dragging meaning that the distance 21 of the foot 20 of the subject during the swing phase 16 is reduced.

Illustrated in solid lines in FIG. 1 is the distance 21 of the foot 20 from the floor 22 during the swing phase 16 according to a target value 24 (“target value of the movement event parameter”). In dotted lines in FIG. 1 is a distance 23 of the foot 20 from the floor 22 according to a current value 26 of the movement event parameter during the swing phase 16, indicating the reduced distance of the foot 20 from the floor 22. In other words, the current movement event of the subject corresponds to the non-corrected movement. Therefore, as can be seen in FIG. 1, the foot lift of the subject during walking requires modulation from the current value 26 to the target value 24.

Aspects and embodiments of the present technology may be used for the modulation of the body part from the current value 26 towards the target value 24.

### System

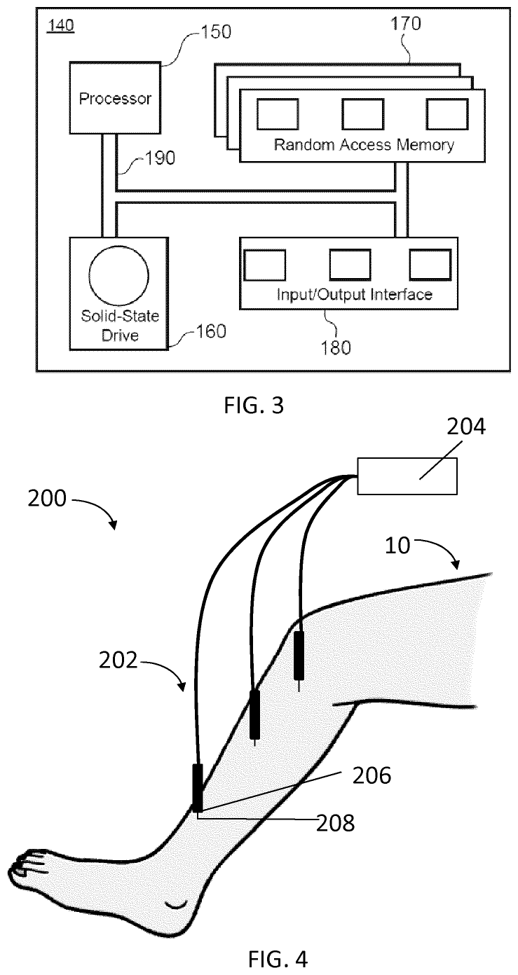

Referring now to FIG. 2, there is shown a system 100 comprising: a computer system 110 operatively connected to a sensor assembly 200 associated with the subject for sensing the current movement event of the leg 10, and a brain stimulation assembly 300 for applying a predetermined modulation signal to tissue of a brain motor region of the subject to cause a modulation of the leg 10 from the current value 26 towards the target value 24 during the swing phase 16 of walking or other movement event.

### Computer System

Turning first to the computer system 110, which is arranged to perform one or more of the following: (i) acquire movement event data of the body part of the subject, the movement event data including the movement event parameter, a trigger point representative of a first time point in the movement event; and the target value, the target value being representative of a second time point in the movement event, (ii) acquire current movement data associated with the current movement event from the sensor assembly 200 and analyze the current movement data including current movement parameter to determine presence of a trigger value of the movement event parameter in the current movement event, and (iii) cause the brain stimulation assembly 300 to apply the predetermined modulation signal in response to identification of the trigger value of the movement event parameter in the current movement data.

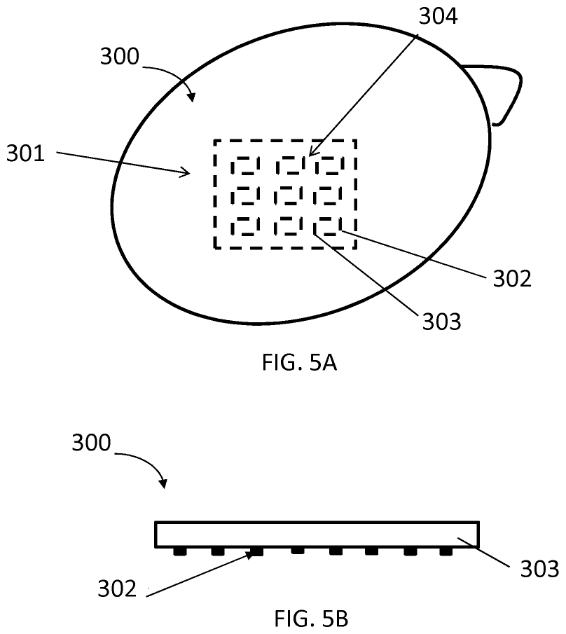

Certain embodiments of the computer system 110 have a computing environment 140 as illustrated schematically in FIG. 3. The computing environment 140 comprises various hardware components including one or more single or multi-core processors collectively represented by a processor 150, a hard drive 160 in this case being a solid-state drive 160, a random access memory 170 and an input/output interface 180. Communication between the various components of the computing environment 140 may be enabled by one or more internal and/or external buses 190 (e.g. a PCI bus, universal serial bus, IEEE 1394 “Firewire” bus, SCSI bus, Serial-ATA bus, ARINC bus, etc.), to which the various hardware components are electronically coupled.

How the processor 150 is implemented is not particularly limited. However, broadly speaking, the processor 150 may be implemented as an electronic circuit configured to perform operations (e.g., processing) on some data provided thereto from a local and/or remote source, and typically, from a memory or some other data stream.

How the solid-state drive 160 is implemented is not particularly limited. However, broadly speaking, the solid-state drive 160 may be implemented as a solid-state storage device that uses integrated circuit assemblies as memory to persistently store data. Nevertheless, it is contemplated that other media can be used as memory to persistently store data, without departing from the scope of the present technology.

How the random access memory 170 is implemented is not particularly limiting. However, broadly speaking, the random access memory 170 may be implemented as a form of computer data storage that stores data and/or machine code (e.g., computer-readable instructions) that is being used by the computing environment 140. The random access memory 170 is arranged to store one or more of: movement event data (set-up data), movement event parameter, movement event parameter as a function of time during the movement event, target value of the movement event parameter, trigger point in the movement event, time points in the movement event, subject data, medical records of one or more subjects, digital anatomy representation data of the one or more of the subjects, and data relating to one or more movement events of one or more subjects, or one or more current movement events. In some embodiments, the above-mentioned data may also be stored in the solid-state drive 160 in a manner that is suitable for being loaded into the random access memory 170.

How the input/output interface 180 is implemented is not particularly limiting. However, broadly speaking, the input/output interface 180 may be implemented so as to allow enabling networking capabilities, such as wire or wireless access, for example. As an example, the input/output interface 180 comprises a networking interface such as, but not limited to, a network port, a network socket, a network interface controller and the like. Multiple examples of how the networking interface may be implemented will become apparent to the person skilled in the art of the present technology. For example, but without being limiting, the networking interface 180 may implement specific physical layer and data link layer standard such as Ethernet™, Fibre Channel, Wi-Fi™ or Token Ring. The specific physical layer and the data link layer may provide a base for a full network protocol stack, allowing communication among small groups of computers on the same local area network (LAN) and large-scale network communications through routable protocols, such as Internet Protocol (IP).

In accordance with at least some implementations of the computing environment 140, the solid-state drive 160 may be configured to store program instructions suitable for being loaded into the random access memory 170 and executed by the processor 150. For example, the program instructions may be part of a library and/or a software application that the computing environment 140 is configured to execute. In another example, as it will become apparent from the description herein below, the program instructions may be part of a software dedicated for modulating movement of a body part of a subject, which program instructions the computing environment 140 may be configured to execute.

In some embodiments of the present technology, the computer system 110 implementing the computing environment 140 may be configured to execute software programs and/or applications for the purpose of aiding the operator of the computer system 110 during movement modulation of the subject, or during a set up phase.

For instance, the computer system 110 may be configured to execute a software experimentation platform. Broadly speaking, software experimentation platforms are typically used for increasing the productivity of the operator during an experimentation control process, improving the quality of the design itself. For instance, when executed by the computer system 110, the software experimentation platform may be used by the operator of the computer system 110 for inter alia display, control, and analysis of current movement data associated with the current movement event from the sensor assembly 200, and the like.

It is contemplated that the computer system 110 may be configured to execute any graphics software that aids an operator of the computer system 110 during the movement modulation or set-up.

In this embodiment, the computing environment 140 is implemented in a generic computer system which is a conventional computer (i.e. an “off the shelf” generic computer system). The generic computer system is a desktop computer/personal computer, but may also be any other type of electronic device such as, but not limited to, a laptop, a mobile device, a smart phone, a tablet device, or a server.

In other embodiments, the computing environment 140 is implemented in a device specifically dedicated to the implementation of the present technology. For example, the computing environment 140 is implemented in an electronic device such as, but not limited to, a desktop computer/personal computer, a laptop, a mobile device, a smart phone, a tablet device, a server, specifically designed for modulating body part movement. The electronic device may also be dedicated to operating other devices, such as one or more of the sensor assembly 200, and the brain stimulation assembly 300.

In some embodiments, the computer system 110 is connected to one or more of the sensor assembly 200 and the brain stimulation assembly 300. In some alternative embodiments, the computer system 110 is implemented, at least partially, on one or more of the sensor assembly 200, and the brain stimulation assembly. In some alternative embodiments, the computer system 110 may be hosted, at least partially, on a server. In some alternative embodiments, the computer system 110 may be partially or totally virtualized through cloud architecture.

In some embodiments, the computer system 110 is distributed amongst multiple systems, such as one or more of the sensor assembly 200, and the brain stimulation assembly 300, the server, and cloud environment. In some embodiments, the computer system 110 may be at least partially implemented in another system, as a sub-system for example. In some embodiments, the computer system 110 may be geographically distributed.

Users of the computer system 110, in certain embodiments, are practitioners and staff of a given clinic. The computer system 110 may also be connected to clinical practice management software which could be used for subject appointment scheduling, inventory management (e.g., for managing stocks of precursor aligners) and other tasks based on the given movement modulation and/or in view of other activities and needs of the clinic. It is also contemplated that the computer system 110 may also be arranged for being used remotely, such as by users of other clinics, for example via server or cloud environment.

As persons skilled in the art of the present technology may appreciate, multiple variations as to how the computing system 110 is implemented may be envisioned without departing from the scope of the present technology.

### Interface Device of the Computer System

Referring back to FIG. 2, the computer system 110 has at least one interface device 192. Broadly speaking, the interface device 192 of the computer system 110 is configured for receiving inputs and/or providing outputs to the operator of the computer system 110. In the embodiment of FIG. 2, the interface device 192 includes a display 194 (such as a screen, for example) for providing a visual output to the operator of the computer system 110.

The visual output may include one or more images pertaining to the movement event, the current movement event, the sensor assembly 200, the brain stimulation assembly 300, and the modulation of the movement of the body part. Other data related to the subject may also be included in the visual output, for example measurements (e.g., leg lift), geometry (e.g., swing phase angle) and identifiers (e.g., body part identifier, subject identifier). The visual output may also include visual data pertaining to operation to any one of the sensor assembly 200, and the brain stimulation assembly 300.

The interface device 192 may also comprise a keyboard 196 and/or a mouse 198 for receiving inputs from the operator of the computer system 100. The interface device 192 may include, in certain embodiments, other devices for providing an input to the computer system 110 such as, without limitation, a USB port, a microphone, a camera or the like. The interface device 192 may comprise a tablet, a mobile telephone, or any other electronic device.

In some embodiments, the interface device 192 may be configured to implement the computing environment 140 of FIG. 3 for processing inputs and/or outputs for the operator of the computer system 110. Put another way, the interface device 192 of the computer system 110 may comprise some or all components of the computing environment 140, without departing from the scope of the present technology.

### Sensor Assembly

The sensor assembly 200 is arranged to sense physiological data associated with the subject, and more specifically for sensing physiological data relating to movement of the body part of the subject.

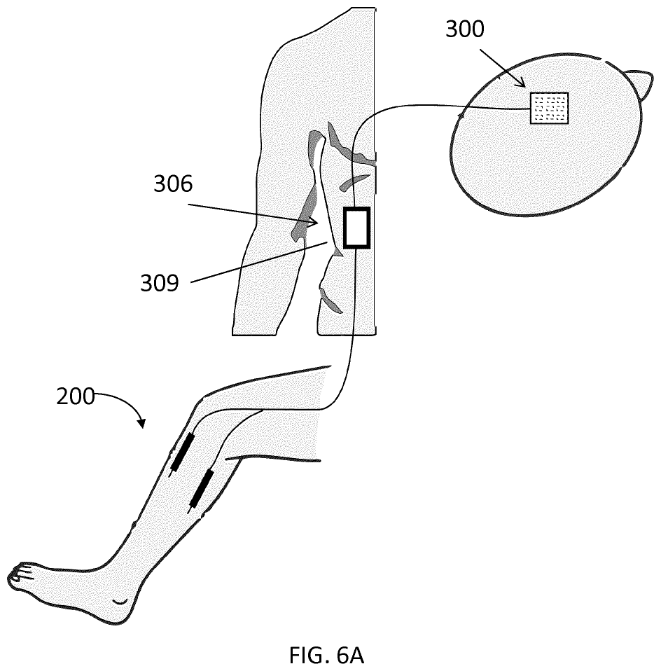

In the embodiment of FIG. 4 and relating to modulating the foot lift of the subject during gait, the sensor assembly 200 includes an intramuscular electromyograph comprising a plurality of electrodes 202 contactable with a muscle of the leg 10 and arranged to sense electrical signals of the muscle, and an electromyograph processor 204 arranged to receive and process the electrical signals. Each electrode 202 of the array of electrodes 202 is a needle electrode comprising a needle 206 with a distal end 208 arranged to be inserted through the skin and into the muscle of interest. The needle 206 may comprise fine wire(s).

In other embodiments (not shown), the sensor assembly 200 includes a surface electromyograph which differs from the intramuscular electromyograph in that the electrodes 202 are arranged to sense electrical signals of the muscle through the skin. In this case, the electrodes 202 are positioned on the skin over the muscle of interest. The muscle of interest may comprise one or more muscles relating to, or associated with, the subject's foot lift (“movement event to be modulated”).

In yet other embodiments (not shown), the sensor assembly 200 comprises an accelerometer associated with the body part for sensing position information relating to the movement of that body part. For example, the accelerometer may comprise a wearable device around the ankle or other leg part of the subject. The accelerometer measures proper acceleration forces.

In yet other embodiments (not shown), the sensor assembly 200 comprises an inertial unit associated with the body part for sensing movement of the body part of one or more of a specific force, an angular rate, and a magnetic field. The inertial unit comprises one or more of an accelerometer, a gyroscope, and a magnetometer. For example, the inertial unit may be incorporated in a wearable device around the ankle or other leg part of the subject.

In yet further embodiments (not shown), the sensor inertial unit comprises a motion detector for detecting movement of the body part of the subject. The motion detector may comprise an infrared detector, an ultrasound detector, or a radiofrequency detector.

In yet other embodiments (not shown), the sensor assembly 200 comprises an imaging device, such as a camera or a video, for capturing images of the movement of the body part. The sensor assembly may include a computer vision analysis module for determining a contour of the imaged body part using one or more image processing or filtering steps. The image processing steps are selected from one or more of image pre-processing, image enhancement, image segmentation, image compression, image restoration, image representation, image recognition, image labelling, image format conversion and the like.

In other embodiments (not shown), the sensor assembly 200 comprises a kinetic marker associated with the body part together with a detector for detecting movement of the kinetic marker.

In yet further embodiments (not shown), the sensor assembly 200 comprises an electroencephalograph for detecting electrical activity in the brain. This can be used to capture brain intention of the movement. Other devices for sensing the current movement event data of the subject are within the scope of the present technology.

### Brain Stimulation Assembly

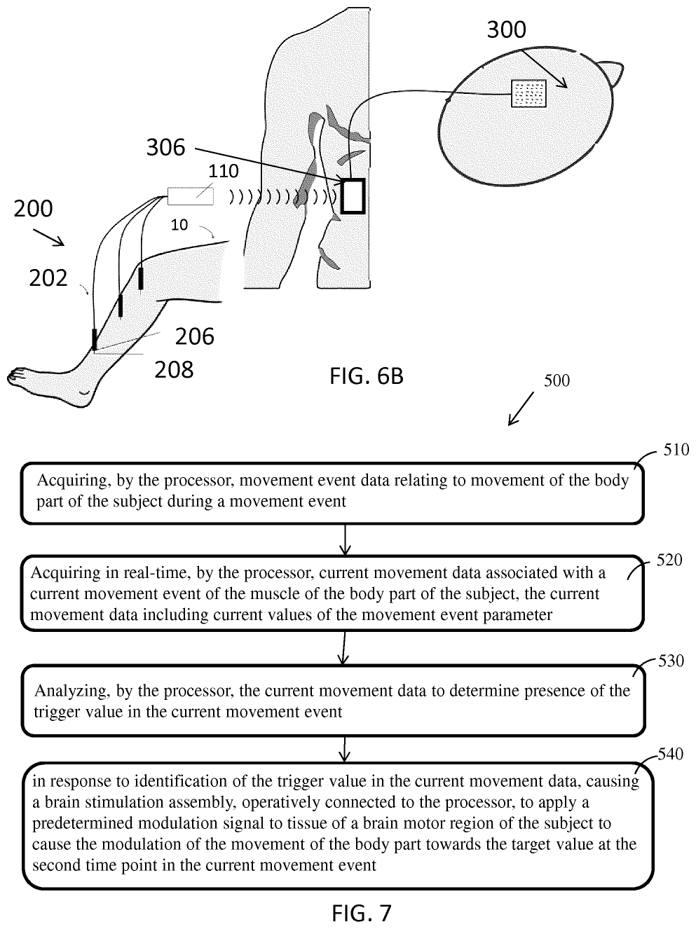

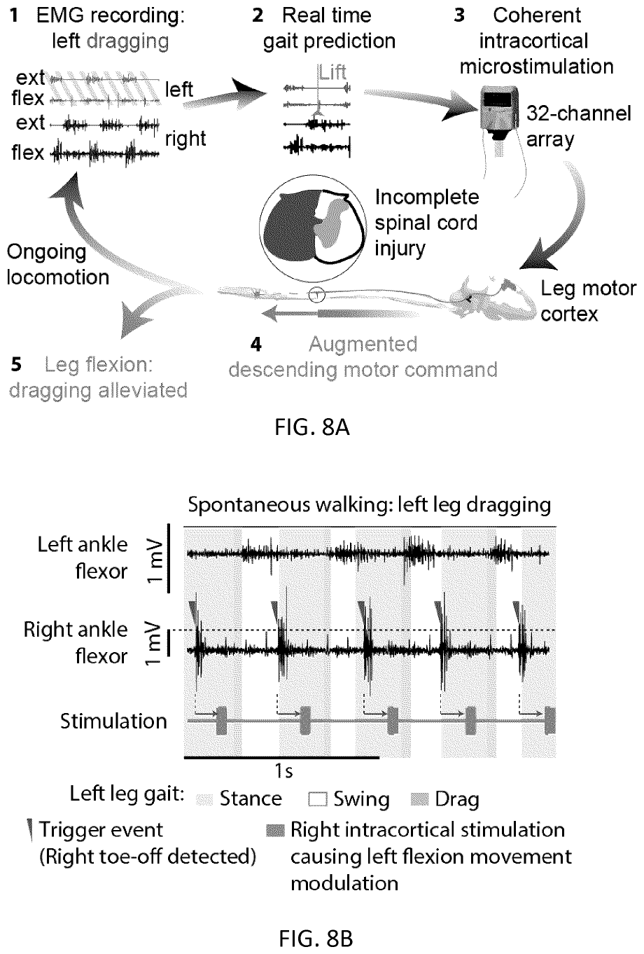

Turning now to the brain stimulation assembly 300 for applying the predetermined modulation signal to brain tissue 301 of the subject. In the embodiments illustrated in FIGS. 5A and 5B, the brain stimulation assembly 300 comprises an array of neural-electrodes 302 extending from a device body 303 for applying an electrical signal to the brain tissue 301. In certain embodiments, the brain tissue 301 is of the brain motor region. The brain tissue may comprise cortical tissue of a cortical motor region 304 of the brain.

As known to persons skilled in the art, the cortical motor region 304 is located approximate the rear portion of the frontal lobe, and is divided into two main areas. The main motor cortex is a thin band along the central sulcus. The subject may undergo a motor function mapping for placement of the array of electrodes 302. Advantageously, cortical stimulation is less invasive than deep brain stimulation and with fewer associated risks.

Each electrode 302 of the array of electrodes 302 has an electrically conductive portion 305 in contact with the brain tissue 301. As illustrated, the electrically conductive portion 305 of the electrodes 302 is contacted directly with an exposed portion of the cortical motor region 304. This is referred to as cortical electrical stimulation. The electrodes 302 may extend from the device body 303 to a length sufficient to allow the electrodes 302 to contact the cortical motor region 304. The device body 303 may be fully implantable or partially implantable, and have the electrodes 302 extending therefrom.

In certain embodiments, the brain stimulation assembly 300 is embodied as a fully implantable or at least partially implantable device which is functionally connected to the sensor assembly 200 and/or to the computer system 110.

In certain embodiments, at least a portion of the system 100 is embodied as at least one implantable device which is sized and shaped to be implanted in the subject, such as subcutaneously. The implantable device is made of a sterilisable, biocompatible material.

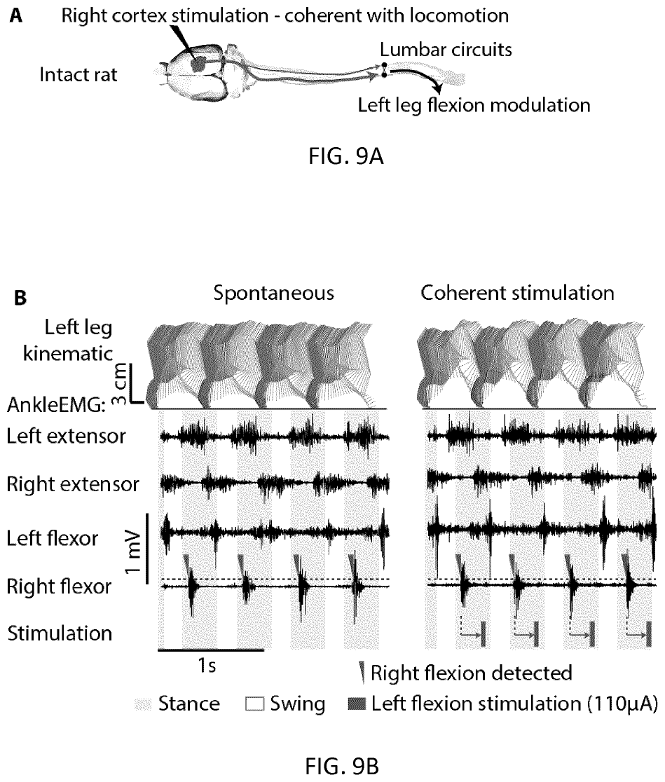

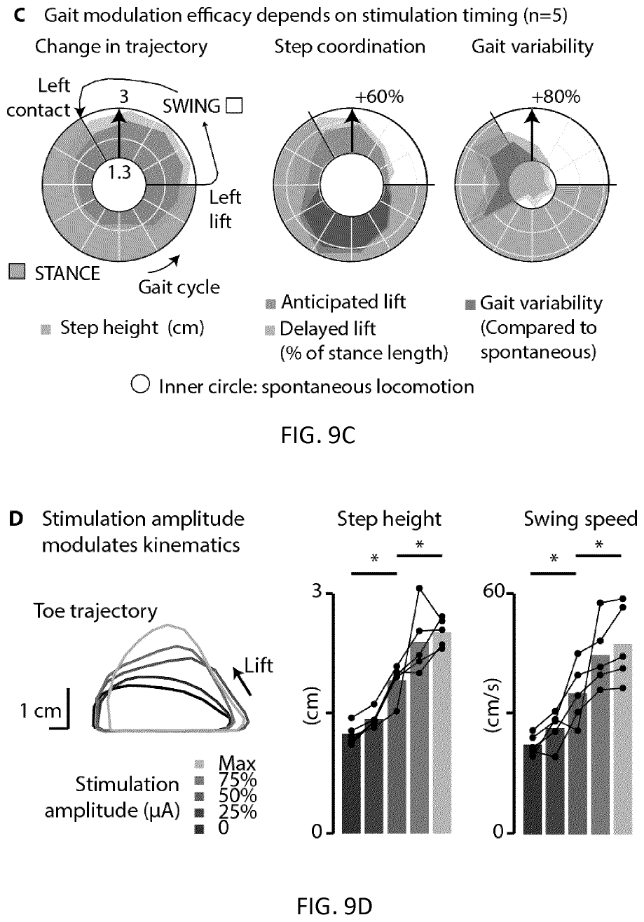

In the embodiment of FIG. 6A, at least a portion of one or more of the computer system 110, the sensor assembly 200, and the brain stimulation assembly 300 is embodied as an implantable processor device 306. The implantable processor device 306 is functionally connected to the sensor assembly 200 and to the brain stimulation assembly 300. The implantable processor device 306 can be positioned subcutaneously on the subject's torso and comprises a body 309.

The implantable processor device 306 comprises at least a portion of the processor 150 of the computer system 110 housed within the body 309. It can be connected subcutaneously to the sensor assembly 200 which in certain embodiments is in the form of an intramuscular electromyograph (FIG. 6A). In other embodiments (not shown), the implantable processor device 306 embodies at least part of a processor associated with one or both of the sensor assembly 200 and the brain stimulation assembly 300.

In the embodiment of FIG. 6B, the implantable processor device 306 includes an electrical pulse generator for creating stimulation waveforms and pulse trains transmitted to the electrodes 302. In other embodiments, the brain stimulation assembly 300 receives stimulation signals generated by an external stimulator (not shown). In yet other embodiments, the electrical pulse generator is housed within the implantable device body 303.

In other words, in certain embodiments, the brain stimulation assembly 300 is shared across the device body 303 including the electrodes 302 contactable with the brain tissue (FIG. 5B), and the implantable processor device 306. The implantable processor device 306 can be connected transcutaneously, wired or wireless, to the computer system 110 which in turn is connectable to the sensor assembly 200.

In certain embodiments, a power supply is housed within the body portion 303 of the brain stimulation assembly 300 or the body 309 of the implantable processor device 306 for providing power to the electrodes 302 or the processor. In other embodiments, one or both of the implantable processor device 306 or the brain stimulation assembly 300 is powered through an external power supply via a cable (not shown).

In certain embodiments, the implantable processor device 306 includes a receiver for receiving instructions from an external processor for applying the modulated signal. In this case, the instructions may be transmitted through any suitable communication network.

In certain embodiments, the brain stimulation assembly 300 includes sensors for detecting electrical signals in the brain of the subject and for transmitting them to a receiver external to the brain stimulation assembly 300. In this respect, at least a portion of the sensor assembly 200 may also be housed within the body portion 308 of the brain stimulation assembly 300.

To summarize, the brain stimulation assembly 300 is arranged to deliver the modulation signal one or more of: intracranially, epidurally, subdurally, and transcranially.

In other embodiments, the brain stimulation assembly 300 comprises a deep brain stimulation device, such as the one described in U.S. Pat. No. 5,683,422. The deep brain stimulation device includes an intracranial electrode.

In further embodiments, the brain stimulation assembly 300 comprises an electromagnetic induction assembly for delivering magnetic signals to the brain. The assembly is composed of a magnetic coil and a stimulator. The stimulator runs changing electric currents through the magnetic coil, causing localized stimulating electrical currents within the brain. This is referred to as Transcranial Magnetic Stimulation (TMS).

In yet other embodiments, the brain stimulation assembly 300 comprises an endovascular stent for delivering electrical signals in the cortical motor region or deep brain motor regions of the brain of the subject. The stent is equipped with an array of electrodes. The electrically conductive portion of the array of electrodes is deployed through endovascular surgery in a blood vessel running in proximity or within a portion of the cortical motor region 304. Advantageously, endovascular cortical stimulation is much less invasive than epidural, subdural and intracortical stimulation and with fewer associated risks.

### Modulation Signal

In certain embodiments in which the modulation signal is an electric signal, the body part is the leg 10, and the movement event to be modulated is the foot lift during the swing phase 16 of gait, the predetermined modulation signal is a biphasic pulsed signal comprising 200 μs/phase, at an emission frequency of 300 Hz, having a length of about 100 ms, and an amplitude of about 40 μA.

In other embodiments, the pulsed signal may be one or more of: cathodic, anodic or biphasic. Each pulse is about 50 to about 400 μs/phase. A frequency of the pulse of the predetermined modulation signal is about 100 Hz to about 400 Hz. A length of the predetermined modulation signal is about 40 to about 500 ms. An amplitude of the pulse is about 5 μA to about 2 mA. The predetermined modulation signal comprises a chain of any number of pulses, such as 14 pulses in 40 milliseconds. Each of these parameters is selected between a minimum value sufficient to elicit muscle contractions and a maximum value comfortable to the subject. Example values are: biphasic pulses 200 μs/phase, emitted at 300 Hz, in 100 ms—long trains, 20 μA for upper limb, 40 μA for lower limb.

### Communication Network

In some embodiments, the computer system 110 is connectable to the sensor assembly 200 and/or the brain stimulation assembly 300 via a communication network 400. In some embodiments, the communication network 400 is the Internet and/or an Intranet. Multiple embodiments of the communication network may be envisioned and will become apparent to the person skilled in the art of the present technology.

In some embodiments, the computer system 110 is connectable to the sensor assembly 200 and/or the brain stimulation assembly 300 via the processor 150. In some other embodiments, the computer system 110 may be directly connected to the sensor assembly 200 and/or the brain stimulation assembly 300. In some alternative embodiments, the computer system 110 or the computing environment 140 is implemented, at least partially, on the sensor assembly 200 and/or the brain stimulation assembly 300. In yet some alternative embodiments, the computer system 110 may be hosted, at least partially, on a server. In some alternative embodiments, the computer system 110 may be partially or totally virtualized through a cloud architecture.

As mentioned above, the system 100 may also comprise the communication network 400. In some embodiments of the present technology, the communication network 400 is the Internet. In alternative non-limiting embodiments, the communication network can be implemented as any suitable local area network (LAN), wide area network (WAN), a private communication network or the like. It should be expressly understood that implementations for the communication network are for illustration purposes only.

The communication network 400 may provide a communication link (not separately numbered) between one or more of the computer system 110, the sensor assembly 200, the brain stimulation assembly 300, and the interface device 192. How the communication network 400 is implemented will depend on how the computer system 110, the sensor assembly 200, the brain stimulation assembly 300, and the interface device 192 are implemented. Merely as an example and not as a limitation, in those embodiments of the present technology where the computer system 110 is implemented as a wireless communication device such as a smartphone or a tablet, the communication link can be implemented as a wireless communication link. Examples of wireless communication links include, but are not limited to, a 3G communication network link, a 4G communication network link, and the like. In some embodiments of the present technology, the communication network 400 may allow the computer system 110 to provide and/or acquire information from external/remote computer systems. For example, the communication network 400 may communicatively couple the computer system 110 with computer systems of other operators and/or of other entities, such as clinics.

### Method

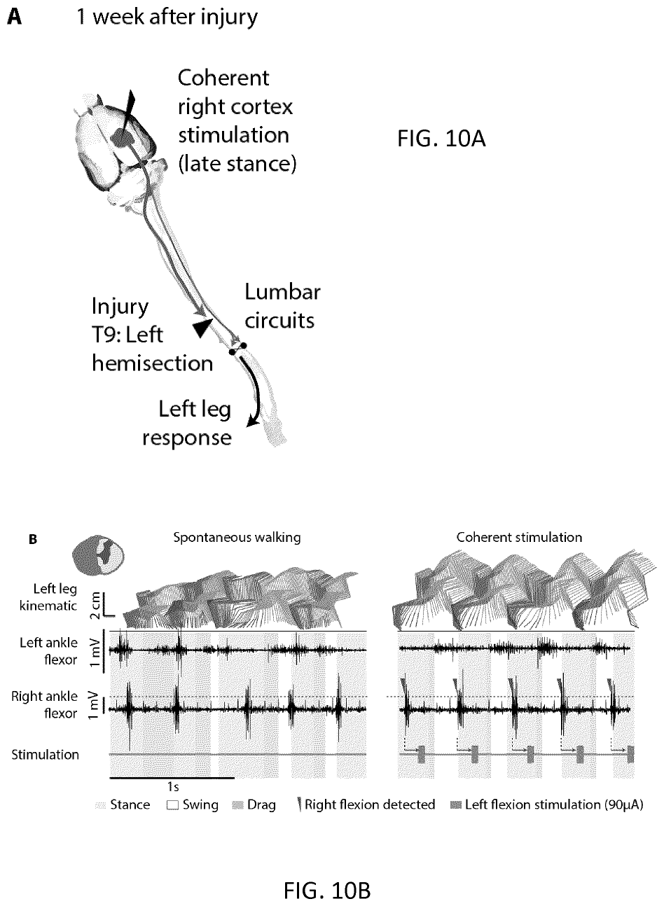

With reference now to FIG. 7, in certain embodiments the computer system 110 is configured to execute a method 500 for modulating the body part of the subject, and more specifically, in certain embodiments, to modulating the body part during the movement event 12 from the current value 26 towards the target value 24 of the movement event parameter. The method 400 will now be described in further detail below.

STEP 502: Acquiring, by the Processor, Movement Event Data Relating to Movement of the Body Part of the Subject During a Movement Event

At step 502, the computer system 110 acquires movement event data relating to movement of the body part of the subject during the movement event. The movement event data includes (i) at least one movement event parameter requiring modulation, (ii) a trigger value representative of a first time point 28 in the movement event, and (iii) the target value 24 representative of a desired movement at a second time point 30 in the movement event, the second time point 30 occurring after the first time point 28. Some or all of the movement event data may be predetermined, and acquired by the processor before subsequent steps of the method 500. This may be considered as a “set-up” stage of the method 500. The subsequent steps may be performed in real-time.

In the embodiment of modulation of foot lift of the subject as illustrated in FIG. 1, it is desired to increase the foot lift of the subject during the swing phase 16, so in other words an upregulation of a current motor output of a leg muscle of the patient is desired.

In this situation, the second time point 30 is associated with a point in time during the movement event when the heel is at a maximum distance from the floor 20.

The first time point 28 is associated with one or more of: an onset of the toe-off phase 14, an onset of the swing phase 16; and a time point between the heel-strike phase 18 and the onset of the next toe-off phase 14.

The trigger value representative of the first time point comprises any value(s) defining the first time point. For example, any one or more of the onset of the toe-off phase 14, the onset of the swing phase 16; and the time point between the heel-strike phase 18 and the onset of the next toe-off phase 14 may be defined by activation of one or more specific muscles in the leg 10. The trigger value may thus be considered as a predetermined marker associated with the first time point in the movement event. The marker may comprise a pattern or a set of patterns. The first time point may be a muscle activation (e.g. contraction).

The target value 24 relates to a desired movement of the body part and can be defined in terms of an increase in foot lift, or a predetermined numerical value e.g. 10 cm lift from the floor 22. The target value can be determined in any suitable manner. For example, the target value 24 can be determined according to a current value of the movement event parameter at the second time point. This may be particularly the case for subject's in which the contra-lateral body part is not affected by a movement disorder,

STEP 504: Acquiring in Real-Time, by the Processor, Current Movement Data Associated with a Current Movement Event of the Muscle of the Body Part of the Subject, the Current Movement Data Including Current Values of the Movement Event Parameter

In Step 504, which may occur in real-time, the current movement data associated with the current movement event is acquired. In certain embodiments, this step 504 comprises acquiring the current movement data from the sensor assembly 200.

As noted above, the current movement data may comprise electromyographic signal data from muscles associated with the body part during walking and representative of contraction and relaxation events relating to the muscles. The current movement data may include the current value of the foot lift of the subject. The current movement data may be associated with the body part requiring modulation, and a contra-lateral body part of the subject.

The method 500 may further comprise determining a difference between the current value of the maximum foot lift and the target value of the foot lift. If there is no difference, the method can stop, or an alert is issued to a user of the system 100. If there is a difference, between the current value of the maximum foot lift and the target value 24, the method 500 may further comprise determining the difference and using this difference to adjust the modulation signal.

STEP 530: Analyzing, by the Processor, the Current Movement Data to Determine Presence of the Trigger Value in the Current Movement Event

In step 530, the processor 150 analyzes or monitors the current movement data to determine the presence of the trigger value. In certain embodiments, the processor 150 compares the current movement data with the trigger value (“marker”) to identify the presence of the trigger value in the current movement event. In embodiments where the trigger value is a signal pattern, the step 530 of determining the presence of the trigger value may comprise a pattern recognition analysis. In certain embodiments, the trigger value may comprise a single value.

STEP 540: In Response to Identification of the Trigger Value in the Current Movement Data, Causing a Brain Stimulation Assembly, Operatively Connected to the Processor, to Apply a Predetermined Modulation Signal to Tissue of a Brain Motor Region of the Subject to Cause the Modulation of the Movement of the Body Part Towards the Target Value at the Second Time Point in the Current Movement Event

In certain embodiments, the method step 540 comprises applying the predetermined modulation signal to the cortical motor region. The modulation signal can be a pulsed electrical signal as described above.

In certain embodiments, the processor 150 is arranged to cause the brain stimulation assembly 300 to apply the predetermined modulation signal at a predetermined time from identifying the trigger value in the current movement event. The predetermined time may be between about 0 seconds and 1 second from identifying the trigger value in human subjects.

Alternatively, the processor 150 is arranged to cause the brain stimulation assembly 300 to apply the predetermined modulation signal until the target value 24 of the movement is detected.

The method 500 further comprises, in certain embodiments, determining, for the given subject, the predetermined modulation signal, the determining the predetermined modulation signal comprising applying a preliminary modulation signal to the tissue of the brain motor region, and adjusting one or more of: an amplitude, a frequency, a duration, and a pulse duration of the preliminary modulation signal until the target value 24 in the second time point 30 in the movement event is obtained.

Stated another way, the obtaining the predetermined modulation signal for the subject comprises; applying a preliminary modulation signal to the brain motor region of the subject; acquiring preliminary movement data associated with a preliminary movement event of the muscle of the body part of the subject, the preliminary movement data including a value of the movement event parameter at the second time point; adjusting one or more of: an amplitude, a frequency, a duration, a pulse duration, and an interval time of the preliminary modulation signal; and determining that the preliminary modulation signal is the predetermined modulation signal when the value of the movement event parameter at the second time point is equivalent to the target value.

The method 500 may further comprise tuning the predetermined modulation signal for the subject, the tuning the predetermined modulation signal comprising: adjusting one or more of: an amplitude, a frequency, a duration, a pulse duration, and an interval time of the predetermined modulation signal responsive to a measured value of the movement event parameter at the second time point.

In certain embodiments, the method 500 comprises executing daily the steps of acquiring in real-time, by the processor 150, the current movement data associated with the current movement event of the muscle of the body part of the subject; the analyzing, by the processor, the current movement data to determine presence of the trigger value in the current movement event; and in response to identification of the trigger value in the current movement data, causing the brain stimulation assembly to apply the predetermined modulation signal to tissue of a brain motor region of the subject to cause the modulation of the muscle of the subject movement towards the target value from the trigger value in the current movement event.

Practice of the disclosure will be still more fully understood from the following examples, which are presented herein for illustration only and should not be construed as limiting the disclosure in any way.

### EXAMPLES

The following examples are illustrative of the wide range of applicability of the present invention and are not intended to limit its scope. Modifications and variations can be made therein without departing from the spirit and scope of the invention. Although any method and material similar or equivalent to those described herein can be used in the practice for testing of the present invention, the preferred methods and materials are described.

### Example 1

### Acquiring Movement Event Data

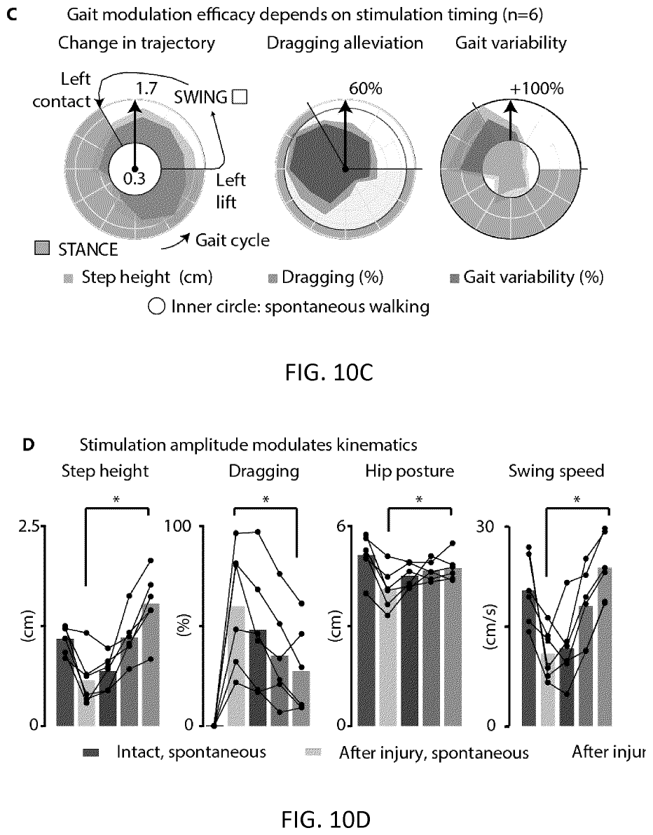

Examples 1-10 concerned a system whereby ongoing locomotor phases were monitored through online processing of electromyographic (EMG) activity from leg muscles of rats. Each leg's lift phase was detected from its ankle flexor activation (FIGS. 8A-B).

The movement event to be modulated was a step. The intramuscular electromyographic activity is movement event data. The trigger value was obtained by analyzing the electromyographic data. The brain stimulation assembly applied intracortical stimulation to the leg motor cortex, causing the modulation of leg flexion and the alleviation of leg dragging. FIG. 8B shows electromyographic activity of left and right ankle flexors. Activation of the right ankle flexor is detected as a trigger event to apply cortical stimulation with a fixed delay of 150 ms that corresponds to a second time point that shortly precedes the leg lift movement to be modulated.

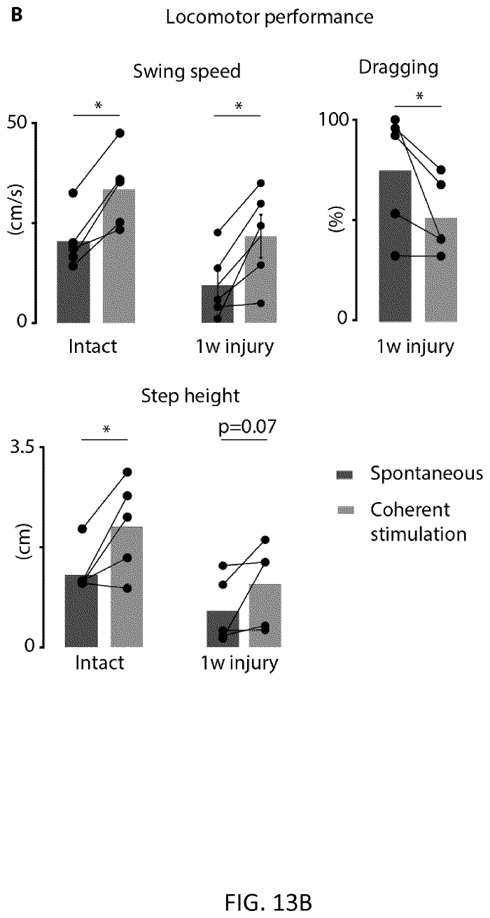

Electrodes of an EMG assembly (“sensor assembly”) were implanted in the left and right ankle flexor and extensor muscles of rats which had an incomplete spinal cord injury (SCI) (“movement disorder”). Movement event data of the rats was acquired and processed using an EMG processor communicable with the electrodes. The rats were then caused to walk on a treadmill in quadrupedal and bipedal stance, the latter by using a horizontal bar for forelimb support (FIG. 13A). A total of 30 parameters quantifying kinematic features were batch-computed for each gait cycle. EMG signals were sampled at 12 kHz and filtered online (bandpass, 70-700 Hz) using a real-time BioAmp processor. Whenever the rectified signal from a selected muscle crossed a manually selected threshold, a gait event was detected (e.g. toe-off phase, swing phase, and heel-strike phase). Thresholds for EMG activity were commonly set at 30-70% of the peak EMG activity during a gait cycle.

The extent of the spinal cord injury on the movement event was determined as follows. Hind limb performance was scored using the Martinez scale. Skilled locomotor control of the rats was determined by acquiring images of the rats (100 frames/s) over a horizontal ladder (130 cm long; rungs of 3 mm spaced by 2 cm). Performance was scored as the percentage of foot-faults made over the number of total consecutive steps. Five trials per rat consisted in a total of roughly 50 steps. During treadmill walking (23 cm/s), six reflective markers were positioned over the iliac crest, trochanter (hip), condyle (knee), malleolus (ankle), fifth metatarsal (foot) and fourth toe tip (limb endpoint) of the rats. Kinematics were captured (119.2 Hz) and offline-processed using custom semi-automated detection software. Each trial consisted in 10 consecutive steps performed under stable kinematics.

It was determined that the rats had foot drag during the swing phase and this was identified as the movement parameter requiring modulation. The expected timing of all intermediate gait phases was predicted using fixed time delays (0-240 ms).

### Example 2

### Brain Stimulation Assembly

A craniectomy was opened over the right hindlimb motor cortex of the rats of Example 1. A 3×2 mm section of dura was removed. Stereotaxically, a matrix of intracortical wires was lowered onto cortical layer V (Dorso-ventral: 1.45 mm). The 32-channel array consisted of 4 columns spaced by 0.375 mm and 8 rows spaced by 0.250 mm. The most antero-medial site was lowered at coordinates [Posterior: 1.1, Lateral: 1.3] mm from bregma. The wires, acting as electrodes for delivering an electric signal to the brain tissue of the rats, in communication with a control device for controlling delivery of the electric signal was the brain stimulation assembly.

### Example 3

### Predetermined Modulation Signal

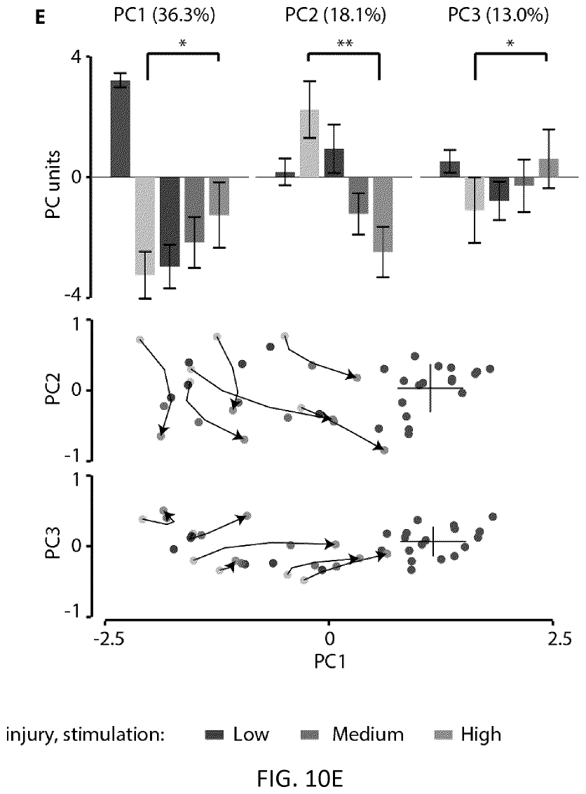

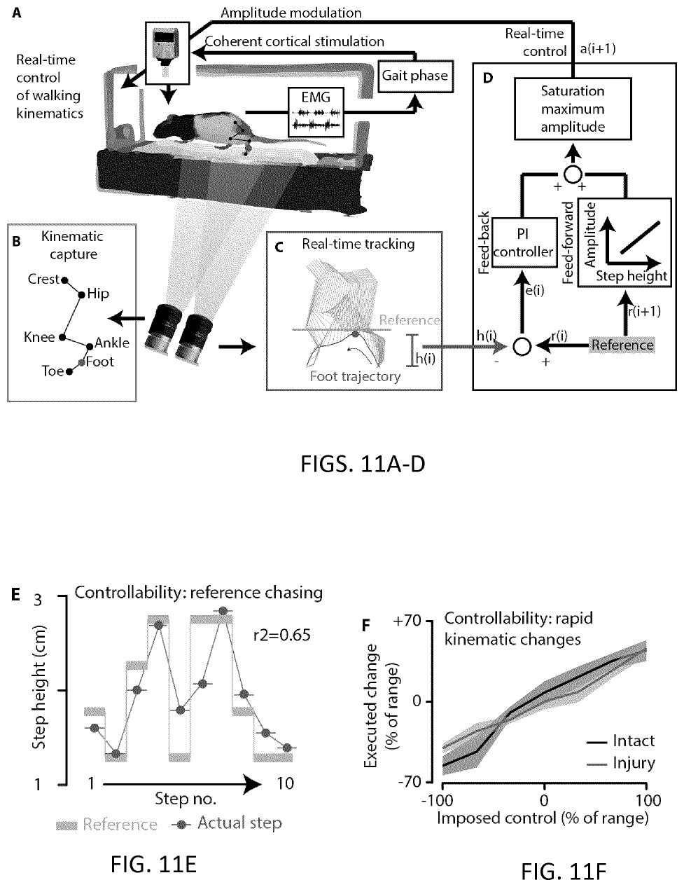

A range of amplitudes of the predetermined modulation signal to be applied by the brain stimulation assembly of Example 2 was defined, with the lower value corresponding to a minimum signal amplitude resulting in visible muscle twitch (20-50 μA) and the upper value to approximately 90% of the maximum comfortable value for each subject (80-300 μA). During stimulation timing characterizations (FIGS. 9C and 10C) the left and right ankle flexors EMG were used for gait event synchronization. The stimulating channel with the strongest distal component and the lowest motor threshold was chosen within the implanted array of the brain stimulation assembly.

Stimulation was delivered with a different timing at each trial (0-240 ms from detection, steps of 40 ms, randomly permutated). Modulation signal delivery was considered to be “coherent” with locomotion when it was delivered in late left stance. In all experiments this phase was detected using the right flexor EMG with a fixed delay of 120-160 ms and confirmed by video recordings. The predetermined modulation signal was applied in real-time within cycles of 24 kHz. Synchronization events triggered delivery of a 40 ms train of intracortical microstimulation (ICMS) (“modulation signal”) with a specified delay (40 ms train with 330 Hz pulse frequency, biphasic, cathodic first, 200 μs/phase, 50 μs inter-phase interval).

FIGS. 9A-D illustrate intracortical stimulation delivery in intact rats in which right cortex stimulation modulates left leg flexion via descending projections. FIG. 9B is a stick diagram and electromyographic activity during spontaneous locomotion and coherent stimulation, triggered during late stance to modulate left leg flexion. FIG. 9C shows phase-dependent effects of stimulation. Stimulation delivery during swing and late stance is associated with maximal modulation of leg flexion. Anticipated or delayed lifts are negotiated by stimulus timing before or after normal foot-off timing. FIG. 9D shows that kinematic variables describing the swing trajectory are linearly modulated by increasing intracortical modulation amplitudes, according to certain embodiments of the present technology.

### Example 4

### Real-Time Control of the Predetermined Modulation Signal

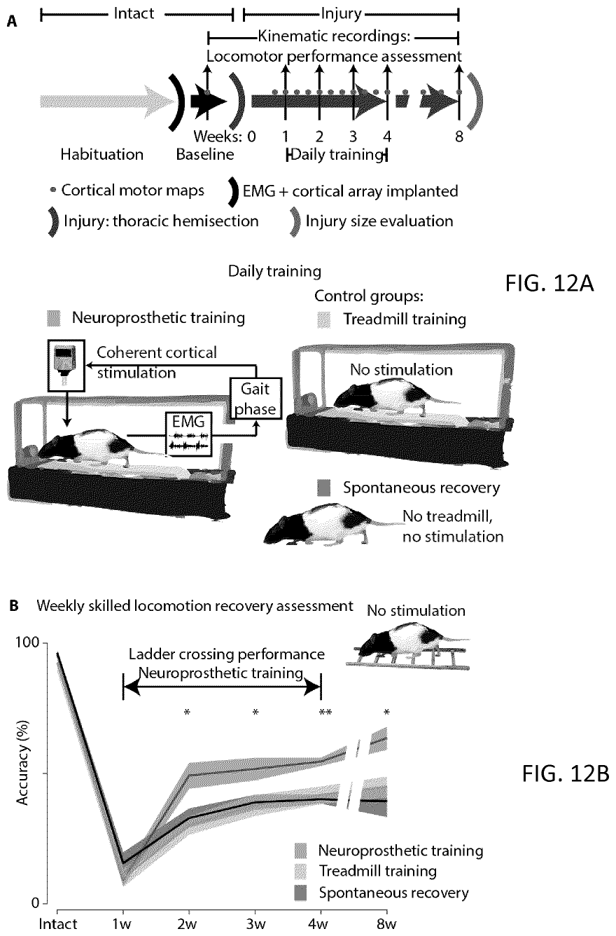

To allow feed-back control of leg kinematics, a soft-real-time system was implemented (FIG. 11A). A camera stream (50 Hz) was processed in real-time to detect the trajectory of a foot marker (positioned slightly above the fifth metatarsal for visibility). At every foot-strike (detected when foot trajectory crossed manually-tuned horizontal and vertical thresholds) the previous step height was compared to a reference value. The error was fed to a PI controller and summed to a linear feed-forward component. The overall controller determined the amplitude of the predetermined modulation signal delivered at the next gait cycle. The feed-forward model was tuned so that the maximum and minimum expected step height for each subject corresponded to the functional range identified for stimulation amplitudes. Stimulation was then saturated to a maximum comfortable value. Stimulation timing remained constant at all gait cycles. Real-time control experiments consisted of sequences of 20-35 consecutive steps with solid behavior. At every step the reference changed within 4 levels (randomly permutated). Imposed reference changes were thus discretized at ±[0, 33, 66, 100]% of range.

### Example 5

### Neuroprosthetic Training

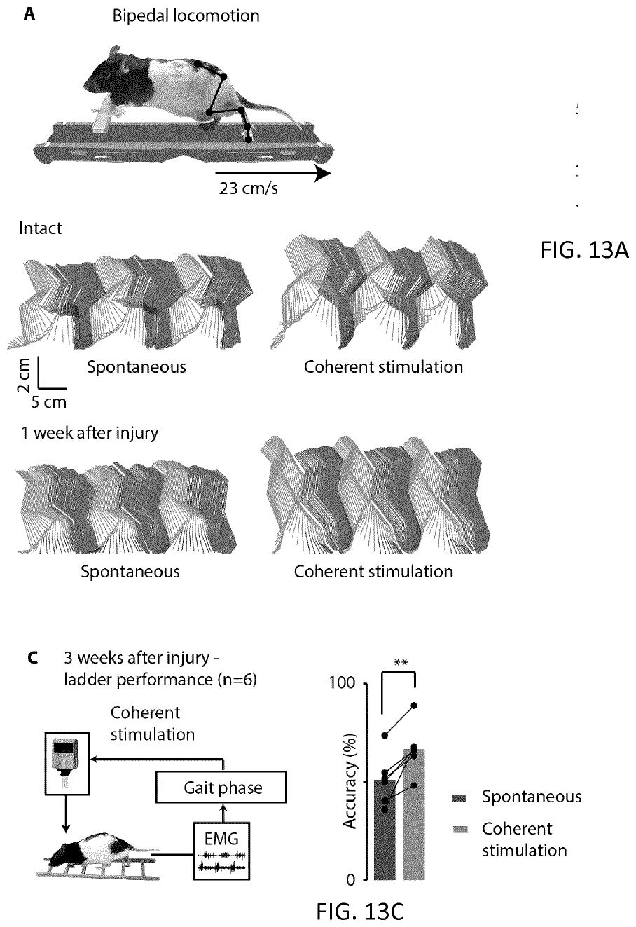

After electrode implantation of the brain stimulation assembly and characterization of intact baseline performance, three groups of n=6 rats underwent thoracic hemisection SCI. Between week 1 to week 4 after SCI, rats were engaged to locomotor training with three different protocols (FIG. 12A). The first group (Neuroprosthetic training) received daily neuroprosthetic stimulation during treadmill locomotion. Stimulation amplitude was set to about 70% of the functional range. Rats performed 30 minute sessions consisting of series of about 30 steps and 5-10 s pauses, and received positive reinforcement (food rewards). The second group (control treadmill training) followed the same protocol without neurostimulation. A third group (control spontaneous recovery) did not perform daily treadmill walking. All groups received weekly kinematic assessment 3 times/week. At week 4 therapy was discontinued, rats were mapped weekly and their ladder performance was tested at week 8.

### Example 6

### Results: Modulation of Locomotion

The expected timing of all intermediate gait phases was predicted using fixed time delays (0-240 ms). Short-train intracortical microstimulation (40 ms, 330 Hz) was delivered at different time points along the gait cycle (FIG. 8A-B) through an electrode chosen from the array implanted in the right leg cortex. The channel was selected by exhaustive search, as the one generating the strongest left ankle flexion. Stimulation amplitude varied as described across experiments within a functional range. During stable treadmill locomotion, stimulation of the right hindlimb motor cortex (FIG. 9A) generated descending motor commands that were behaviorally expressed as enhanced contralateral leg flexion movements (FIG. 9B).

Changes in leg trajectory and locomotor behavior were assessed in n=5 intact rats and found to be stimulation-timing-dependent. Although stimulation delivered in all gait phases produced at least a slight increase in foot clearance above the ground, the largest change in step height was obtained by stimuli delivered between right and left foot contact (p=0.007, peaking at +139±35% of spontaneous levels). This gait hemi-cycle is composed of left swing preparation and execution. When stimulation was delivered before the natural timing for left lift it resulted in an anticipated lift movement (p=0.002, down to 47±5%, FIG. 9C). Conversely, stimulation delivered after left lift had the opposite result. Indeed, during stable locomotion, the subjects negotiated predictable time-locked stimulation by modifying their gait rhythms. This resulted in delayed left lift movements (p=0.03, up to 147±11%, FIG. 9C). When stimulation was delivered around right lift, rats did not seem to succeed negotiating gait timing with stimulation delivery. This resulted in large variability in gait rhythms compared to all other phases (p=0.05, up to +156±39%, FIG. 9C). Thus, intracortical microstimulation was considered to be ‘coherent’ with locomotor behavior when it is delivered in proximity to the contralateral leg lift.

The effects of changes in amplitude of coherent intracortical stimulation on leg flexion was investigated. On all tested subjects, step height (p=0.03, up to 211±14%) and swing speed (p=0.03, up to 214±24%) modulated with a linear trend (linear fitting r²: 91±4%) for increasing stimulation amplitudes (FIG. 9D).

### Example 7

### Results: Modulation of Locomotion to Alleviate a Movement Disorder

Lateral spinal hemisection is the incomplete SCI model that, for a given expected ablation of half of descending fibers, maximizes the loss of motor connectivity between the contralateral cortex and the ipsilesional leg. Acutely, hemisection results in unilateral complete hindlimb dragging. Approximately one week after hemisection, rats recover alternated hindlimb locomotion, affected by severe dragging.

In n=6 rats, lateral spinal hemisection model was used to induce unilateral complete hindlimb dragging for testing the effects of intracortical microstimulation on locomotion after injury (FIG. 10A). One week after SCI, coherent intracortical microstimulation successfully enhanced the leg flexion movement, which in turn alleviated locomotor deficits in all tested animals (FIG. 10B). FIG. 10B is a stick diagram and electromyographic activity during spontaneous locomotion and coherent stimulation, triggered during late stance to modulate left leg flexion. Consistently with results obtained in intact rats, effects in step height were maximal when stimulation was delivered during swing preparation and execution (p=0.002, up to 133±39%, FIG. 10C). FIG. 10C shows phase-dependent effects of stimulation. Stimulation delivery during swing and late stance is associated with maximal modulation of leg flexion. Dragging alleviation is optimal for stimulation delivered in late stance, shortly anticipating the foot flexion movement to be modulated. Stimulation was most effective in reducing dragging when delivered in the swing preparation phase (p=0.003, down to 50±8%, FIG. 10C). When stimulation was delivered in counter-phase to this interval, it increased gait rhythm variability (p=0.01, up to 135±23%, FIG. 10C).

The key kinematic parameters that coherent intracortical microstimulation immediately reversed multiple locomotor deficits associated with SCI (FIG. 10D) was investigated, thus effectively acting as an intracortical neuroprosthesis. FIG. 10D shows that kinematic variables describing the swing trajectory are linearly modulated by increasing intracortical modulation amplitudes. Delivered during swing preparation and across a range of amplitudes, it increased step height (p=5E-4) in a linear fashion (linear fitting r²: 86±8%), reduced dragging (p=2E-4), enhanced posture (p=7E-4, +24±7%) and swing speed (p=5E-4, +134±28%). A multi-variate analysis (30 variables, FIG. 10E) of gait patterns was performed and compared to those of intact subjects. It was found that locomotion shaped by the intracortical neuroprosthesis was closer to intact rats (p=0.05, FIG. 10E). FIG. 10E represents 30 kinematic variables in principal component space (Principal Component Analysis). Coherent stimulation brings locomotion closer to intact subjects.

### Example 8

### Results: Controllability of Leg Motor Output by Intracortical Neuromodulation

An additional closed-loop control system was designed (FIG. 11A). The inner loop illustrates electromyographic readings trigger detection by pattern recognition and timed cortical stimulation, as in FIG. 8A, 9A, 10A.

The outer loop illustrates camera recordings (FIG. 11B-11C) processed to track foot trajectory and compare it to a desired reference. Online video recordings (FIG. 11C) were fed to a controller composed of a proportional-integral (PI) and a feedforward linear component (FIG. 11D). At every step, the controller tested whether the leg trajectory could match a desired reference by imposing changes in stimulation amplitude. Thus, the leg trajectory was guided across 4 kinematic levels (FIG. 11E). The controller comprised two parallel feed-back and feed-forward elements induces changes to the stimulation amplitude and control locomotor output. In both intact and SCI rats (n=5 each) the performance of the overall online system was evaluated. The EMG-based detection and stimulation delivery was very reliable in subjects with and without SCI (99±3% and 99±2% correct detections respectively). Video detection of gait cycles was highly reliable, although slightly inferior after injury (99±2% and 92±6% correct detections respectively). Online tracking fidelity of step height matched 68±7% and 76±7% variance of that captured with the high-speed camera and offline processed, respectively. The system controlled 62±7% and 51±10% of leg endpoint trajectory during ongoing locomotion (FIG. 11E). In FIG. 11E, step height was modulated to follow an arbitrary reference across 4 kinematic levels. FIG. 11F shows that rapid changes in control trajectory are immediately followed by locomotor output, with 50% inertia, in intact rats and rats with spinal cord injury. By tracking leg responses to rapid changes in reference leg trajectory, the experiment allowed to reveal the level of immediate controllability of leg output. 54±7% (intact) and 46±4% (SCI) of the imposed control changes were immediately converted into trajectory changes.

### Example 9

### Results: Coherent Intracortical Neuromodulation Fosters Chronic Recovery of Leg Motor Control After SCI

The intracortical neuroprosthesis relies on augmented volleys of neuronal activation, generated in the motor cortex, that descend to the lumbar circuits resulting in stronger locomotor output. It was hypothesized that tapping into the cortex's own leg control networks through daily sessions of neuroprosthetic training could manipulate cortico-spinal transmission by activity-dependent mechanisms and chronically increase cortical control of movement after SCI.

n=18 rats were divided in three groups (FIG. 12A). After SCI, one group walked 30 min/day on a treadmill, while receiving coherent cortical stimulation (40 ms trains of 330 Hz pulses; 30-150 μA current amplitude, set at about 75% of the maximum comfortable value for each subject). A second group performed the same protocol, with no stimulation. A third group was left to spontaneous recovery. The effects of this neuromodulation protocol was tested with weekly assessment of skilled locomotor performance, obtained during ladder walking—an untrained task—while no stimulation was delivered. Rats that received daily neuroprosthetic intervention, as in Example 7 (FIGS. 10A-E), overperformed both other groups (p=0.007,) and as soon as 1 week after the beginning of stimulation (p=0.01, FIG. 12B). Performance was retained 4 weeks after neuroprosthetic intervention was discontinued (p=0.01). No difference emerged between rats that received daily treadmill locomotion sessions and rats that underwent spontaneous recovery.

FIG. 12B illustrates locomotor performance of rats from the three groups during ladder crossing, which is a skilled locomotor task. Although some animals received stimulations during training, no stimulation was performed during this test, in order to assess spontaneous voluntary motor control. The group that was trained with cortical stimulation delivered with this method to modulate leg flexion during walking outperforms all other groups as soon as week 2 and long after training is discontinued.

### Example 10

### Results: Modulation of Locomotion to Alleviate a Movement Disorder During Bipedal Locomotion and Ladder Crossing

It was confirmed that the effects of the intracortical neuroprosthesis were retained during different locomotor tasks. During bipedal locomotion (FIG. 13A-B) in n=5 intact rats stimulation increased swing speed (p=0.03) and step height (p=0.02). One week after SCI, it alleviated leg dragging (n=5, p=0.04). Tested three weeks after injury, during ladder crossing (FIG. 13C), rats made less foot slip errors when coherent stimulation was delivered (n=6, p=0.006). FIG. 13B illustrates kinematic variables relative to FIG. 13A. FIG. 13C illustrates that the method increases ladder walking accuracy in rats with spinal cord injury.

### Example 11

### Epidural Cortical Stimulation

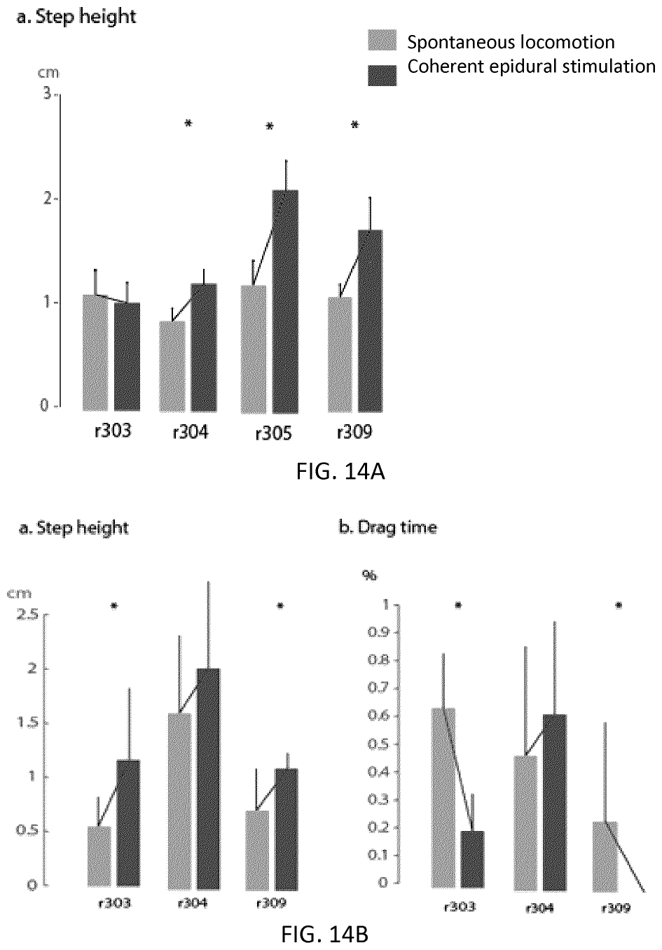

Rats with a movement disorder (SCI, as described above) had the modulated signal applied by epidural cortical stimulation by the brain stimulation assembly and in a manner coherent with the gait. The movement performance of the rats with and without the epidural cortical stimulation (40-100 ms stimulation, 330 Hz, 600-2700 μA) were compared. FIG. 14A shows that epidural cortical stimulation significantly increased step height in 3 out of 4 intact rats. FIGS. 14B and 14C shows that epidural cortical stimulation significantly increased step height and reduced dragging in 2 out of 3 rats. Stimulation was not effective at any time point on r304. Post-mortem analysis showed that due to insufficient fixation the stimulating electrodes had lost adherence to the dura mater in this subject.

### Example 12

### Ipsilateral Modulation of Locomotion

Most connections between the motor cortex and spinal motoneurons which control movement are crossed, meaning that stimulating the left cortex will primarily affect the right leg. However, some of these connections are not crossed. Accordingly, stimulating the left cortex could be used to produce changes in left leg trajectory. Here, the left cortex of a rat was stimulated during walking (40 ms trains, 330 Hz, 30-150 μA) and changes in left hip position were obtained, reducing low posture deficits due to spinal cord injury. Increasing stimulus amplitude results in stronger extensions and higher hip position.

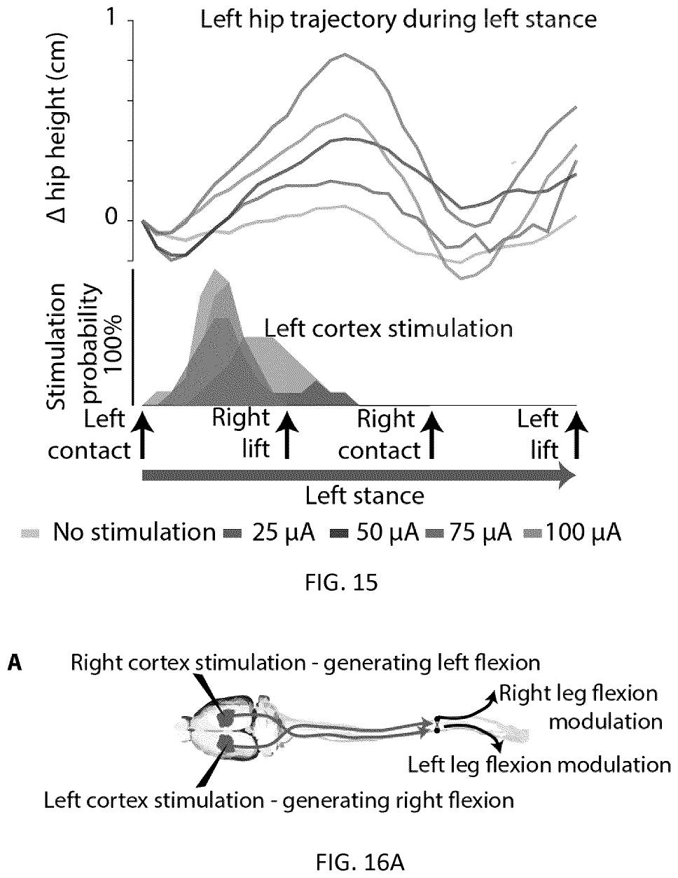

These results (FIG. 15) show that stimulation of the left cortex can be used to modulate movement of the left side of the body, as well as stimulation of the right cortex can be used to modulate movement of the right side of the body. Stimulation of each cortex can be used to control both sides of the body. In FIG. 15 the vertical trajectory of the left hip is displayed along the gait cycle time. Stimulation of the left cortex was triggered by electromyographic activity on the left ankle flexor. An appropriate delay of 150 ms from the trigger allowed stimulation delivery to be performed in the instants preceding right leg lift. The system allowed to increase the left hip position, reducing low posture deficits.

### Example 13

### Bilateral Modulation of Locomotion

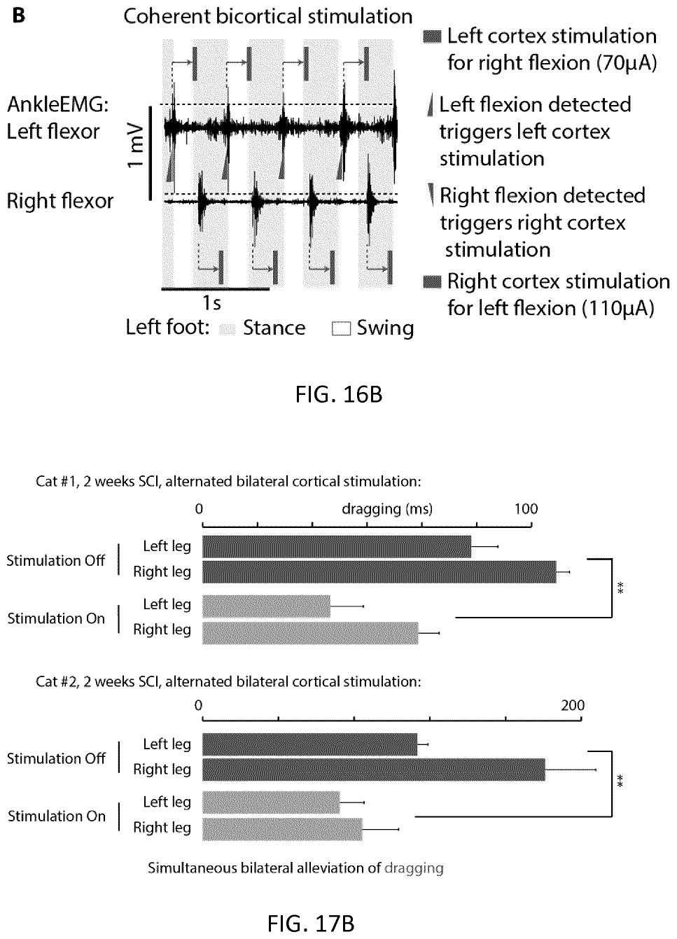

Since stimulation of each cortex would have primary effect on the opposite limb, alternate stimulation of the left and right cortex may be used to improve bilateral movement. In an embodiment, the left ankle flexor EMG of a rat with spinal cord injury was used to detect a first movement (left flexion) and trigger left cortex stimulation (40 ms trains, 330 Hz, 30-150 μA) with an appropriate delay (100-240 ms). The stimulation improved the right leg flexion. In turn, detection of the right leg flexion was used to trigger right cortex stimulation (40 ms trains, 330 Hz, 30-150 μA) with a similar delay. The stimulation improved the left leg flexion (FIG. 16). Stimulation amplitudes are tuned to desired values, which depend on the desired intensity of the modulated movement. FIG. 16A represents a scheme of descending motor commands generated by bilateral cortical stimulation in one rat with incomplete spinal cord injury. FIG. 16B shows electromyographic traces for the left and right ankle flexor muscles. In this example, left and right ankle flexions are detected by the respective electromyographic trace crossing a selected threshold. Stimulation is delivered after a selected delay (160 ms) to slightly anticipate and improve the contralateral leg lift.

In another embodiment, stimulation of the right cortex was used to improve extension on the right leg, while stimulation of the left cortex was used to improve flexion of the right leg. In another embodiment, alternate stimulation of both cortices was used to improve right flexion and left extension (left cortex) and left flexion and right extension (right cortex).

### Example 14

### Bilateral Modulation of Locomotion in a Cat Model

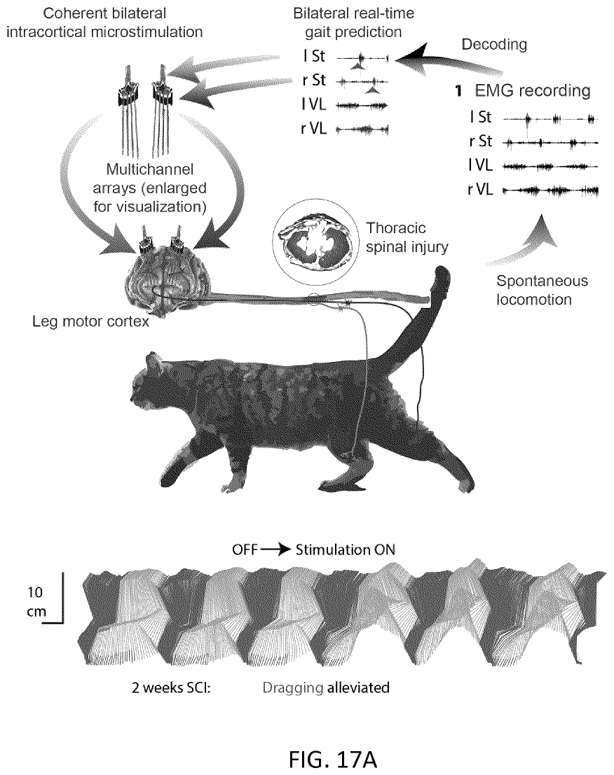

In n=2 cats, alternate intracortical stimulation of the left and right cortex improved bilateral leg movement. The left ankle extensor EMG of a cat with contusive spinal cord injury was used to detect a first movement (left extension) and trigger left cortex stimulation with an appropriate delay (50-200 ms). The stimulation improved the right leg flexion. In turn, detection of the right leg extension was used to trigger right cortex stimulation with a similar delay. The stimulation improved the left leg flexion. Stimulation was 100 ms long (330 Hz), amplitudes were tuned to values 50-500 μA and tuned for maximum effect with no discomfort for the experimental animal. On the first cat, dragging was reduced by 52% and 39% for left and right leg respectively. On the second cat, dragging was reduced by 36% and 56% for left and right leg respectively. T-test: p<0.01 for both cats and both legs (FIGS. 17A and 17B).

It should be expressly understood that not all technical effects mentioned herein need to be enjoyed in each and every embodiment of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A computer-implemented method for modulating a movement of a body part of a subject, the method executable by a processor of a computer system, the method comprising:
acquiring, by the processor, an indication of a movement event of a muscle of the body part of the subject, the movement event defined by at least one movement event parameter, the at least one movement event parameter comprising:
a trigger value representative of a first time point in the movement event,
acquiring in real-time, by the processor, current movement data associated with a current movement event of the muscle of the body part of the subject, the current movement data including current values of the movement event parameter;
analyzing, by the processor, the current movement data to determine presence of the trigger value in the current movement event; and
in response to identification of the trigger value in the current movement data, causing a brain stimulation assembly, operatively connected to the processor, to apply, at a predetermined time from the identification of the trigger value in the current movement event, a predetermined modulation signal to a tissue of a brain motor region of the subject to cause the modulation of the movement of the body.

2. The method of claim 1, wherein the at least one movement event parameter further comprises a target value representative of a desired movement at a second time point, the second time point occurring after the first time point, and the target value comprises an up-regulation of a current motor output of the muscle of the body part of the subject.

3. The method of claim 1, wherein the motor output comprises a contraction of the muscle of the body part, or a series of contractions of the muscle of the body part.

4. The method of claim 1, wherein the body part is a leg, the current movement event is a phase of a gait, and the motor output is a distance of lift of a leg of the subject during the phase of the gait.

5. The method of claim 4, wherein the trigger value and/or the first time point is associated with one or more of:
an onset of a leg lift or a contact phase of the gait,
a swing phase of the leg; and
a time point between a contact phase of a foot of the leg and a contact phase of a contralateral foot of a contralateral leg.

6. The method of claim 1, wherein the body part is one or more of: an arm, a leg, a neck, a trunk, a hand, a foot, a finger, and a toe, and wherein the movement event is one or more of: walking, running, gripping, breathing, standing, and swallowing.

7.-10. (canceled)

11. The method of claim 1, the processor is arranged to cause the brain stimulation assembly to apply the predetermined modulation signal until the target value of the movement is detected.

12.-14. (canceled)

15. The method of claim 1, wherein the predetermined modulation signal comprises a pulsed electrical signal.

16.-19. (canceled)

20. The method of claim 15, wherein the body part is a lower limb, and the predetermined modulation signal is a biphasic pulsed signal comprising 200 μs/phase, at an emission frequency of 300 Hz, having a length of about 100 ms long, and having an amplitude of one of about 20 μA and about 40 μA.

21.-22. (canceled)

23. The method of claim 1, wherein the current movement data comprises one of:
electromyographic signal data associated with the muscle of the body part of the subject, the method further comprising acquiring the electromyographic signal;
image data associated with the current movement, the method further comprising obtaining the image data;
brain signal data associated with the subject, the method further comprising obtaining the brain signal data; and
acceleration data associated with the current movement, the method further comprising obtaining the acceleration data.

24.-27. (canceled)

28. The method of claim 1, further comprising determining, for the subject, the predetermined modulation signal, the determining the predetermined modulation signal comprising applying a preliminary modulation signal to the tissue of the brain motor region, and adjusting one or more of: an amplitude, a frequency, a duration, and a pulse duration of the preliminary modulation signal.

29.-31. (canceled)

32. The method of claim 1, further comprising executing daily the steps of acquiring in real-time, by the processor, the current movement data associated with the current movement event of the muscle of the body part of the subject; the analyzing, by the processor, the current movement data to determine presence of the trigger value in the current movement event; and in response to identification of the trigger value in the current movement data, causing the brain stimulation assembly to apply the predetermined modulation signal to tissue of a brain motor region of the subject to cause the modulation of the muscle of the subject movement.

33. The method of claim 1, further comprising tuning the predetermined modulation signal for the subject, the tuning of the predetermined modulation signal comprising: adjusting one or more of: an amplitude, a frequency, a duration, a pulse duration, and an interval time of the predetermined modulation signal responsive to a measured value of the movement event parameter at the second time point.

34. A system for modulating a movement of a body part of a subject, the system comprising:
a sensor assembly, operably communicable with a processor of a computer system, arranged to sense in real-time, current movement data associated with a current movement event of a muscle of the body part of the subject;
a brain stimulation assembly, operably communicable with the processor of the computer system, for applying a predetermined modulation signal to tissue of a brain motor region of the subject; and
the computer system having the processor arranged to execute a method comprising:
acquiring an indication of a movement event of the muscle of the body part of the subject, the movement event defined by at least one movement event parameter, the at least one movement event parameter comprising:
a trigger value representative of a first time point in the movement event, and
acquiring the current movement data from the sensor assembly and analyzing the current movement data to determine presence of the trigger value in the current movement event; and
in response to identification of the trigger value in the current movement data, causing the brain stimulation assembly to apply the predetermined modulation signal to tissue of a brain motor region of the subject to cause the modulation of the muscle of the subject movement towards the target value from the trigger value in the current movement event.

35. The system of claim 34, wherein the sensor assembly comprises one or more of: an electromyograph, a camera, an infrared sensor, a brain signal detector, and an accelerometer.

36. The system of claim 34, wherein the brain stimulation assembly is an implantable device.)

37. (canceled)

38. The system of claim 36, wherein the implantable device is sized and shaped to be positioned subcutaneously in the subject.

39. The system of claim 36, wherein the implantable device includes one or more of:
a power supply,
a transmitter,
a receiver, and
a processor.

40. A method of treatment of a movement disorder in a mammal with an incomplete spinal injury, the method according to claim 1.

41. The system of claim 34, wherein the at least one movement event parameter further comprises a target value representative of a desired movement at a second time point, the second time point occurring after the first time point, and the processor is arranged to cause the brain stimulation assembly to apply the predetermined modulation signal until the target value of the movement is detected.
